(19) United States
(12) Patent Application Publication
(10) Pub. No.: US 2025/0064811 A1
(43) Pub. Date: Feb. 27, 2025

(54) SELF-ADMINISTRATION INJECTION DEVICE FOR RISPERIDONE

(71) Applicant: ORBICULAR PHARMACEUTICAL TECHNOLOGIES PRIVATE LIMITED, Hyderabad (IN)

(72) Inventors: Patel Bhaveshkumar VALLABHBHAI, Hyderabad (IN); Rajesh KINI, Hyderabad (IN); Vignesh MOHAN, Hyderabad (IN)

(21) Appl. No.: 18/724,799

(22) PCT Filed: Dec. 29, 2022
(86) PCT No.: PCT/IB2022/062868

§ 371 Date: Jun. 27, 2024

(30) Foreign Application Priority Data
Dec. 31, 2021 (IN) 202141062125

(51) Int. Cl. A61K 31/519; A61K 9/00; A61K 9/16; A61M 5/20

(52) U.S. Cl. CPC A61K 31/519; A61K 9/0019; A61K 9/1617; A61K 9/1647; A61M 5/20

(57) ABSTRACT

The present invention relates to an self-administration injection device, comprising drug reservoir of long-acting risperidone microspheres in non-aqueous liquid vehicle. The risperidone microspheres are suspended in non-aqueous liquid vehicle such as triglycerides. The self-administration injection device is auto-injector for single dose.

### FIELD OF THE INVENTION

The present invention relates to a self-administration injection device for Risperidone that enables a patient to self-administer Risperidone microspheres suspended in a liquid vehicle.

### BACKGROUND OF THE INVENTION

Long-acting injectable formulations are used for pharmacotherapy as sustained release medication over a period of several days, weeks or even months. Compared to conventional preparations, long-acting injectable formulations has many distinguished advantages related to its long-lasting curative effect as well as its reduced unwanted toxicity, enhanced systemic availability by avoiding first pass metabolism, dosage accuracy and frequency of administration without compromising the effectiveness of the treatment.

Risperidone is an atypical antipsychotic drug and it is benzisoxazole derivative compound. The chemical name is 3-[2-[4-(6-fluoro-1,2-benzisoxazol-3-yl)-1-piperidinyl]ethyl]-6,7,8,9-tetrahydro-2-methyl-4H-pyrido[1,2-a]pyrimidin 4-one. The mechanism of action of risperidone in schizophrenia is unclear. It acts as a strong dopaminergic antagonist and selective serotonin receptor antagonist and therapeutic activity in schizophrenia could be mediated through a combination of dopamine type 2 (D2) and serotonin type 2 (5HT2) receptor antagonism. The clinical effect from risperidone results from the combined concentrations of risperidone and its major active metabolite, 9-hydroxyrisperidone (paliperidone).

Risperidone is commercially available as long-acting injectable formulations under the brand name Risperdal Consta® and Perseris™. Risperdal Consta®, available in dosage strengths of 12.5 mg, 25 mg, 37.5 mg, and 50 mg risperidone, is approved for the treatment of schizophrenia and bipolar disorder as an intramuscular injection administered once every two weeks. It is supplied as a dose pack, consisting of a vial containing the risperidone microspheres, a pre-filled syringe containing diluent. Risperidone is micro-encapsulated in 75:25 polylactide-co-glycolide (PLG) at a concentration of 381 mg Risperidone per gram of microspheres. Perseris™, an extended-release once a monthly injectable suspension for subcutaneous use and available in strengths of 90 mg and 120 mg, indicated for the treatment of schizophrenia in adults. Perseris™ is supplied as a kit which includes two pre-filled syringes: one pre-filled syringe containing a white to yellow risperidone powder in and another pre-filled syringe containing a colorless/N-methyl-2-pyrrolidone.

The Risperdal Consta® Intramuscular injection requires multiple reconstitution steps and oral supplementation at the start of treatment. Intramuscular injections are typically painful and anxiety inducing, in particular to the target population. Perseris®, provided in two syringes which require significant mixing and resuspension prior to administration.

WO2021/059211 discloses auto-injector device configured specifically with sensors and processor. The invention specifically discloses the injector devices configured sensors to gather various data associated with the patient, and at least one processor that can analyse the data gathered by the one or more sensors and adjust delivery of the drug based on the data. WO2021/059211 generically teaches that any of a variety of drugs can be delivered using a drug administration device including risperidone as one of the example of drug from the large list.

WO2018/046718 and WO2020/065272 discloses Injection device comprises a housing, a dose indicator positioned within the housing and a dose selector operatively connectable to the dose indicator. The injection device further includes syringes of all types, devices that contain said syringes such as auto-injectors, pen-injectors, patch injectors and other similar devices. The medicament container comprises a pre-filled syringe or cartridge and generically teaches that medicament selected form the group comprising one of the agent among the list be risperidone.

WO2018/046722 discloses Injection device with dose indicator mechanism. The injection device further comprises medicament container, comprises a pre-filled syringe or cartridge. WO2018/046722 generically teaches that medicament selected form the group comprising one of the agents among the list be risperidone.

WO2021/048817 discloses injectable formulation comprising risperidone, triblock and diblock copolymers wherein the concentration of the risperidone is 250-400 mg/mL and injection volume is 1 mL or less. Injectable formulation presented in a single prefilled syringe (PFS) for subcutaneous administration.

WO2021011896 discloses long-acting composition comprising one of the active agent risperidone, a non-polymeric, non-water soluble high viscosity liquid carrier material (HVLCM) is sucrose acetate isobutyrate, a lactic acid-based polymer is PLGA and solvent is propylene carbonate. Further discloses in some embodiments, administration of provided compositions is accomplished via a pre-filled syringe or an auto-injector.

WO1997044039A1 discloses depot formulation for administration by intramuscular or subcutaneous injection, comprising 9-hydroxy risperidone palmitate ester i.e. Paliperidone palmitate is suspended in pharmaceutically acceptable carrier is water. Further discloses that the 9-hydroxy risperidone fatty acid ester suspended in sesame oil, medium-chain triglyceride (Miglyol™). The Miglyol™ formulations exhibited considerably less systemic and local tolerance than the sesame oil-based formulations and aqueous suspensions of the 9-hydroxyrisperidone palmitate ester outperformed the sesame oil-based formulations.

Antipsychotic medication is the first line treatment for psychiatric diseases or disorders. The route of administration, patient compliance, follow up treatment and cost associated with the treatment have a major role in adherence of pharmacotherapy in psychiatric diseases or disorders. Poor adherence to medication is a significant problem in the treatment of schizophrenia and bipolar disorders, and is a frequent cause of illness relapse, hospitalisation and worsening of the patient's symptoms and function. The commercially available intramuscular injection requires bimonthly hospital visits, with associated costs, time and transportation, which may serve as a barrier to adherence to follow up injections, leading to poor adherence to pharmacotherapy during maintenance-phase treatment of bipolar disorder, exposing patients to a high risk of illness relapses, rehospitalization. The prevention of relapse in psychiatric diseases or disorders remains an unmet need in the art and improvements in this area can have tremendous impact on morbidity, mortality and quality of life, as well as direct and indirect health care costs.

There is unmet need in the art for long-acting risperidone formulations with patient compliance, the formulation easy to prepare and administer. Further subcutaneous administration of long-acting injectable formulations of risperidone remains one of the challenge due high dosage and maintenance dosage are needed for longer duration of action in treating psychiatric diseases or disorders such as schizophrenia and bipolar disorder.

For addressing these problems, there is unmet need to develop a self-administration injection device for subcutaneous administration comprising long-acting injectable composition of risperidone. The present invention provides self-administration auto-injector device for subcutaneous administration. The present invention provides long-acting risperidone microsphere formulation suspended in non-aqueous vehicle provided in auto-injector device in a low injection volume, with an injectable viscosity for subcutaneous self-administration by patient himself or their caregiver.

Further present invention provides auto-injector device makes self-administration easier for patients or their caregivers compared to manual injection with prefilled syringes hence facilitating adherence to the regimen. Self-administration offers psychological benefits over administration by healthcare professionals, including improved self-esteem, ease of use, patient compliance. This improved treatment experience may lead to better adherence, improved therapeutic outcomes, also optimizing the use of healthcare resources and associated cost. Therefore, a need exists for self-administration of long-acting formulation with the help of auto-injector device. Furthermore, self-administration of long-acting injection of risperidone by auto-injector device of present invention reduce patient medication taking burden, improved medication adherence and prevention of relapse, more accurate dosing, regularity of treatment and simplified medication regimen.

### SUMMARY OF THE INVENTION

The present invention relates to a self-administration injection device for the administration of risperidone microsphere suspended in liquid vehicle; wherein the said injection device is auto-injector for single dose.

The present invention relates to a self-administration auto-injector device for the administration of risperidone microsphere suspended in non-aqueous vehicle such as medium chain triglycerides.

The present invention relates to self-administration injection kit comprises a container having reservoir comprises risperidone microspheres suspended in medium chain triglycerides and instructions for use. Further the container is a single dose auto-injector.

### DETAILED DESCRIPTION OF THE INVENTION

The present invention relates to a self-administration injection device for the administration of long-acting microsphere of active pharmaceutical ingredients or pharmaceutically acceptable salts thereof which are suspended in liquid vehicle.

The present invention relates to a self-administration injection device for the administration of long-acting microspheres of risperidone or pharmaceutically acceptable salts thereof which are suspended in vehicle; wherein the said injection device is auto-injector for single dose.

The term “active pharmaceutical ingredient” or “drug” or “API” refers to a substance that has a physiological effect when ingested or otherwise introduced into the body, in particular or a chemical substance used in the treatment, cure, prevention, or diagnosis of disease or used to otherwise enhance physical or mental well-being.

The “active pharmaceutical ingredient” or “drug” or “API” is Risperidone or its derivative or its metabolite or optical isomer, diastereomer, enantiomer, tautomer, or physiologically acceptable salt and solvates thereof.

The term “pharmaceutically-acceptable salts” refer to derivatives of the disclosed compounds wherein the parent compound is modified by making acid or base salts, solvate, hydrate, esters and the like thereof. Pharmaceutically acceptable salt forms of compounds provided herein are synthesized from the parent compound which contains a basic or acidic moiety by conventional chemical methods.

The term “injection device” as used herein refer to a device intended for the injection of a medicament to the body and includes devices configured for various delivery methods, such as intradermal, subcutaneous, intramuscular, intravenous, intraperitoneal, intrathecal, epidural, intracardiac, intraarticular, intracavernous, and intravitreal, which may include via a cannula, catheter or similar device. The term includes syringes of all types, devices that contain said syringes such as auto-injector device for subcutaneous administration.

The term “self-administration injection devices” as used herein includes administration of the preparation or composition or formulation by a person himself or caregiver and/or without the intervention of another person. Self-administrable devices may include auto-injector, single dose auto-injector, single dose reusable auto-injector, dual chamber cartridge with auto-injector, auto-injector with prefilled syringe and the like. The term “caregiver” or “caretaker” as used herein includes healthcare professional such as doctors, nurses or non-healthcare professionals such as family member, friends, relatives who assists in administration of injection to the patient.

The term “microspheres” or “microparticles” as used herein is small spherical or spherical-like particles or powder consist of drug dissolved and (or) dispersed homogeneously throughout a polymer material, with a particle size ranging in 1-1000 μm, and generally made up of biodegradable and biocompatible polymers.

The “Biodegradable polymers” as used herein “biodegradable” is meant a material that shall degrade by bodily processes to products readily disposable by the body and should not accumulate in the body. The products of the biodegradation should also be biocompatible with the body. The “biocompatible” is meant not toxic to the human body, is pharmaceutically acceptable, is not carcinogenic, and does not significantly induce inflammation in body tissues.

“Carrier” or “vehicle” or “solvent” or “diluent” as used herein refers to pharmacologically inert materials that provide a more or less fluid matrix, suitable for parenteral drug administration. Carriers, vehicles or diluent useful herein include any such materials known in the art, which are nontoxic and do not interact with other components of a pharmaceutical formulation or drug delivery system in a deleterious manner.

The terms “composition” or “formulation” or “preparation” refer to preparations comprising risperidone or pharmaceutically acceptable salts thereof in the form of microspheres or microsphere powder or microsphere powder in vehicle, in a form suitable for administration.

The term “parenteral administration” as used herein is intramuscular, intraperitoneal, intra-abdominal, subcutaneous, intravenous and intraarterial, intradermal, intravitreal, intracerebral, intrathecal, epidural administration. In some embodiments, administration is subcutaneous.

The term “risperidone sensitive diseases or disorders” includes but not limited to psychiatric diseases or disorders such as schizophrenia, schizoaffective disorders, bipolar disorder, bipolar mania, bipolar disorder and irritability, bipolar I acute manic or mixed episodes, irritability associated with autistic disorder, major depressive disorder, non-schizophrenic psychoses, management of behavioral disturbances and psychosis in patients suffering from dementia, depression, anxiety, adjunct to antidepressants in treatment-resistant depression, symptomatic management of aggression or psychotic symptoms in patients with severe dementia of the Alzheimer type.

According to the present invention, microspheres comprises risperidone or pharmaceutically acceptable salts thereof dissolved and (or) dispersed homogeneously throughout a biodegradable polymer material. The microspheres particles, wherein particles are in the range of 1000 microns or less and injectable viscosity. In a particular embodiment, the microsphere provides are easily passable through 23-gauge needle.

According to present invention the biodegradable or biocompatible polymers include but not limited to lactide-glycolide copolymer. It is also referred to as poly(lactide-co-glycolide), abbreviated as PLGA. Simply, “PLGA” is a poly lactic acid and poly glycolic acid copolymer. The preferred polymer is the copolymer, poly(d,l-lactide-co-glycolide). It is preferred that the molar ratio of lactide to glycolide in such a copolymer be in the range of from about 60:40 to about 80:20; more preferably the molar ratio of lactide to glycolide in such a copolymer from about 75:25.

According to present invention the active pharmaceutical ingredient is Risperidone in microspheres may be present in the form of any salt thereof. An acid which will form a salt with risperidone comprises an inorganic acid, for example, halogen acid (e.g., hydrochloric acid or hydrobromic acid), nitric acid, sulfuric acid or phosphoric acid; or an organic acid, for example, acetic acid, propionic acid, hydroxy acetic acid, 2-hydroxy propionic acid, pamoic acid, 2-oxo propionic acid, oxalic acid, malonic acid, succinic acid, 2-butenedioic acid, methanesulfonic acid, ethanesulfonic acid, benzenesulfonic acid or toluenesulfonic acid.

The microspheres of the present invention may be prepared by processes disclosed and known in the art, e.g., in U.S. Pat. Nos. 5,792,477, 6,596,316, 6,667,061, 6,379,703.

According to present invention the “carrier” or “vehicle” is preferably pharmaceutically acceptable non-aqueous liquid material. The carrier is preferably generally regarded as safe (GRAS) and approved by regulatory agencies or included in Pharmacopoeia. Furthermore, carrier does not solubilize the polymer, and active pharmaceutical ingredient within the microspheres.

The example of non-aqueous liquid material includes but not limited to triglycerides, preferably medium chain triglycerides (MCT's). The medium chain triglycerides (MCT's) are a type of structurally modified lipid in which three saturated fats with intermediate carbon lengths are linked to a glycerol backbone, forming triacylglycerols or triglycerides with a chain length of C6-C12 (i.e. caproic acid (C6), caprylic acid (C8), capric acid (C10) and dodecanoic acid is sometimes incorporated as a minor component). MCTs are made up of a mixture of medium-chain fatty acids (MCFA's) found in palm kernel oil and coconut oil produced through the hydrolysis and subsequent fatty acid separation. They can also be created through the process of interesterification. MCT's are having a lower melting point, smaller molecular size, molecular weight, are liquid at room temperature, and are less energy dense than triglycerides containing mostly saturated long chain fatty acids. The viscosity, density, and stability of oil vehicles are all determined by the fatty acid makeup.

One embodiment of present invention, provides self-administration injection device, comprising: drug reservoir comprising a liquid injectable composition comprises risperidone microspheres and vehicle, wherein said injection device is a single dose.

Another embodiment provides self-administration injection device, comprising: drug reservoir comprising a long-acting liquid injectable composition comprises risperidone microspheres and non-aqueous vehicle, wherein said injection device is a single dose.

Another embodiment provides self-administration injection device, comprising: drug reservoir comprising a long-acting liquid injectable composition comprises risperidone microspheres and non-aqueous vehicle, wherein said injection device is an auto-injector for a single dose.

The present invention relates to self-administration injection devices such as auto-injector, single dose reusable auto-injector device, dual chamber cartridge with auto-injector, auto-injector with prefilled syringe and the like, comprising a long-acting risperidone microspheres in a pharmaceutically acceptable vehicle.

In one embodiment of present invention provides the self-administration injection device is auto-injector.

In another embodiment of present invention provides the self-administration injection device is auto-injector for single dose.

In another embodiment, the self-administration injection device is single dose auto-injector comprising drug reservoir of long-acting microspheres of pharmaceutically active ingredient in non-aqueous liquid vehicle.

In another embodiment, the self-administration injection device is single dose auto-injector comprising drug reservoir of long-acting risperidone microspheres in non-aqueous liquid vehicle.

In another embodiment of present invention relates to self-administration injection devices is single dose auto-injector comprising drug reservoir of long-acting risperidone microspheres in non-aqueous liquid vehicle such as triglycerides.

In another embodiment of present invention relates to self-administration injection devices is single dose auto-injector comprising drug reservoir of long-acting risperidone microspheres in non-aqueous liquid vehicle such as medium chain triglycerides.

In one embodiment of present invention relates to self-administration injection devices is a single dose auto-injector comprising drug reservoir of long-acting microspheres of pharmaceutically active ingredient in non-aqueous liquid vehicle.

In one embodiment of present invention provides an auto-injector device in various dimensions, designs and components of device assembly.

In another embodiment, an auto-injector device can be customised in different design and configured for different fill volumes for individual dosing requirements.

Another embodiment of present invention an auto-injector device includes housing, a cartridge disposed in housing, drug reservoir, plunger, needle and other associated device assembly parts.

Another embodiment of present invention, an auto-injector device further includes an actuation assembly and said device is digitally configured.

Another embodiment, an auto-injector device in a self-contained, reusable, preset and prefilled cartridge device configured to present a dosing choice to an end-user and to deliver a dose associated with the choice made by the end-user. As such, the auto-injector is configured to be capable of delivering a single dose chosen from a plurality of different doses of a pharmaceutical composition or medicament. In one aspect, the auto-injector is configured to be capable of delivering a single dose chosen from two different doses of a pharmaceutical composition or medicament. In other words, the device is capable of delivering different preset doses. The prefilled device may be a single chamber; wherein single chamber may comprise of the ready to use preparation or multi chamber.

Another embodiment of present invention provides self-administration injection device an auto-injector device may be configured to allow for delivering of one or optionally two, three, four or multiple doses.

Another embodiment of present invention provides self-administration injection device an auto-injector device may be configured to allow for delivering single or optionally multiple doses.

The sterile solution filled cartridge should be loaded into the reusable auto-injector device and ensure proper closure of the device to make ready for dosing. Before initiating next dose ensure the rigid needle shield should be removed or ejected form auto-injector device. After removing the needle shield the recorded dose is administered immediately.

The auto-injector is thereby configured to administer to a patient a pharmaceutical composition stored in a medicament reservoir disposed within it. The medicament reservoir cooperates with the dose selection component so that only the volume of pharmaceutical composition, at a given concentration corresponding to the selected dose is administered to the patient using the device. In one other embodiment, the medicament reservoir is a suitable device such as cartridge or refillable cartridge and the like thereof, incorporated into the auto-injector housing, either permanent or removable.

The auto-injector is configured with the assembly for selection of the dose, either the higher dose or lower, yet already preset dose, is made by some form of manipulation of the injector such as by use of a button, slide, dial, shaking, audible/visible selection, or other input mechanism on the single use, auto-injector. The injection device is calibrated so that only the amount of medicament that corresponds to the end-user dosage selection is dispensed from the reservoir and administered to the patient.

The auto-injector shall contain at least an auto penetration mechanism and an autoinjection mechanism and also contains an auto return mechanism. Auto-injector device of present invention further comprises feedback mechanism provided to the user can be any form of sensory feedback, such as for example visual feedback, auditory feedback, or tactile feedback.

Another embodiment of present invention, an auto-injector device is digitally configured with smart device or smart phone. The smart digital device assembly may display to the user a current status of the injection device to display, for example, information to assist a user with injection technique. The smart device may display on a display screen a scaled force profile to allow a user to monitor progress of an injection event, dose scale, set dose to be injected, dose left in cartridge or like. The calculation of the scaling prior to user display may be performed on the smart device. The smart device may indicate an end of injection event detected by the machine learning algorithm implemented by the processor.

Another embodiment of present invention provides ready to use or ready to administer auto-injector device comprises drug reservoir of long-acting risperidone microspheres suspended in non-aqueous liquid vehicle.

Another embodiment of present invention provides ready to use or ready to administer auto-injector device comprises drug reservoir of long-acting risperidone microspheres suspended in non-aqueous liquid vehicle such as triglycerides, preferably medium chain triglycerides.

Another aspect, the present invention is not limited solely to such an auto-injector in foregoing description. A person skilled in the art understands that various modifications to the described auto-injector may be implemented within the scope of the present disclosure.

One embodiment of present invention, the long-acting risperidone microspheres suspended in non-aqueous liquid vehicle such as triglycerides.

Further embodiment of present invention, the long-acting risperidone microspheres suspended in non-aqueous liquid vehicle such as medium chain triglycerides.

Further embodiment of present invention, the long-acting risperidone microspheres suspended in non-aqueous liquid vehicle such as medium chain triglycerides; wherein said composition presented in self administration auto-injector device.

The pharmaceutical preparation further comprises of excipients such as tonicity adjusting agent, buffering agent, vehicle, carrier, pH modifiers, antioxidants, preservatives, chelating agents, bulking agents, diluents, surfactant, stabilizers and the like or mixtures thereof.

The pharmaceutical preparation, wherein, tonicity adjusting agent include but not limited to mannitol, sucrose, maltose, dextrose, trehalose, glycerin, sodium chloride, potassium chloride or mixtures thereof. Tonicity adjusting agents decrease the haemolysis of blood cells and reduce pain and irritation at the injection site.

Examples of buffering agent include but not limited to acetate, citrate, tartrate, phosphate, benzoate, lactate, gluconate, bicarbonate, organic amines, its salts, hydrates, solvates, or any mixtures thereof.

Examples of vehicle or carriers include but not limited to water for injection, hydroalcoholic solvents like, propylene glycol, polyethylene glycol, ethanol, glycerol, oils, triglycerides and mixtures thereof.

Examples of antioxidants include but not limited to monothioglycerol, L-cysteine, thioglycolic acid, sodium metabisulfite, ascorbic acid, sodium formaldehyde sulfoxylate, sodium bisulfate, butylated hydroxy toluene (BHT), butylated hydroxy anisole (BHA), phenylmercuric nitrate, thiomersal, benzalkonium chloride, benzethonium chloride, phenol, cresol or chlorobutanol.

Examples of preservatives include but not limited to benzyl alcohol, m-cresol, phenol, methyl parabens, propylparaben, butylparaben, chlorbutanol, thiomersal, phenylmercuric salts, or mixtures thereof. The sterile solution of the present invention comprises preservative in amounts sufficient to maintain sterility of the solution in the injection device, throughout the shelf life of the product, which may be exposed to repeated multiple injections. This is because it is possible that the antimicrobial preservative concentration in a given preparation may decrease during the product's shelf life.

Examples of chelating agents include but not limited to ethylene diamine tetraacetic acid (EDTA) and acceptable salts thereof, citric acid, sodium citrate, ethylene glycol-bis(beta-aminoethyl ether)-N,N,N′,N′-tetraacetic acid (EGTA) and acceptable salts thereof, and 8-Amino-2-[(2-amino-5-methylphenoxy) methyl]-6-methoxyquinoline-N,N,N′,N′-tetraacetic acid, tetrapotassium salt (QUIN-2) and acceptable salts thereof and the like or mixtures thereof.

Examples of stabilizers include but not limited to polyols, amino acids, surfactants, poloxamer and its co-polymers; polyols include but not limited to propylene glycol, polyethylene glycol, polypropylene glycol and the like or mixtures thereof; amino acids include but not limited to aspartic acid, glutamic acid, glycine, arginine, lysine, L-histidine and the like or mixtures thereof; surfactants include but not limited to polysorbate-20, polysorbate-80, and the like or mixtures thereof.

The present invention provides microsphere formulation, such that the microspheres are suspended in a non-aqueous pharmaceutically acceptable carrier. The present invention is provided as a single component injectable microsphere formulations comprising Risperidone dispersed in non-aqueous liquid vehicle, the liquid vehicle is triglyceride, more preferably medium chain tri glyceride and microspheres which consist essentially of a poly(lactide-co-glycolide) polymer preferably PLGA 75:25.

In one embodiment of present invention is provided as a kit comprising a medicament container and instructions for use; wherein the container is an auto-injector containing cartridge.

In another embodiment of present invention is provided as a kit comprises a medicament container filled with long-acting risperidone microsphere composition in non-aqueous vehicle and instructions for use; wherein the container is an auto-injector containing cartridge.

In another embodiment of present invention is provided as a kit comprises a medicament container filled with long-acting risperidone microsphere composition in non-aqueous vehicle such as triglycerides; preferably medium chain triglycerides and instructions for use; wherein the container is an auto-injector containing cartridge.

In one embodiment of present invention provides self-administration of injectable device by patient himself or their caregiver.

Antipsychotic medication is the first line treatment for psychiatric diseases or disorders. Long-acting antipsychotic injections are widely used and proven treatment compared to oral medications for individuals with schizophrenia, schizoaffective disorder or bipolar disorder. Risperidone is used in first-line treatment of schizophrenia due to its safety profile and recommendation for medium and long-term treatment. The route of administration, patient compliance, follow up treatment and cost associated with the treatment have a major role in adherence of pharmacotherapy in psychiatric diseases or disorders. The prevention of relapse in schizophrenia remains a health challenge and improvements in this area can have tremendous impact on morbidity, mortality and quality of life, as well as direct and indirect health care costs. Poor adherence to medication is a significant problem in the treatment of schizophrenia and bipolar disorders, and is a frequent cause of illness relapse, hospitalisation and worsening of the patient's symptoms and function. The commercially available intramuscular injection requires bimonthly hospital visits, with associated costs, time and transportation, which may serve as a barrier to adherence to follow up injections, leading to poor adherence to pharmacotherapy during maintenance-phase treatment of bipolar disorder, exposing patients to a high risk of illness relapses, rehospitalization.

In one embodiment, self-administration administration of long-acting injection of risperidone by auto-injector device of present invention reduce patient medication taking burden, improved medication adherence and prevention of relapse, more accurate dosing, regularity of treatment and simplified medication regimen.

In other embodiment, self-administration auto-injector device suitable for parenteral administration such as intramuscular, intraperitoneal, intra-abdominal, subcutaneous, intravenous and intraarterial, intradermal, intravitreal, intracerebral, intrathecal, epidural administration.

In other embodiment, self-administration auto-injector device suitable for intravenous, intramuscular or subcutaneous administration. In other embodiment, self-administration auto-injector device suitable for subcutaneous administration.

In one embodiment of present invention provides self-administration of long-acting risperidone microspheres composition in non-aqueous vehicle via auto-injector by patient himself or their caregiver.

In other embodiment of present invention provides subcutaneous administration of long-acting risperidone microspheres composition in non-aqueous vehicle via auto-injector by patient himself or their caregiver.

In one embodiment, present invention provides the single component long-acting injectable non-aqueous formulation of poorly water soluble active pharmaceutical ingredient microspheres in single use auto-injector device; wherein the microspheres are suspended in a non-aqueous liquid vehicle and easily injectable through 23G needle suitable for parenteral administration.

In one embodiment, the single component long-acting injectable non-aqueous formulation of Risperidone microspheres in single use auto-injector device; wherein the Risperidone microspheres are suspended in a non-aqueous liquid vehicle is triglycerides and easily injectable through 23G needle suitable for subcutaneous administration.

In one embodiment, the single component long-acting injectable non-aqueous formulation of Risperidone microspheres in single use auto-injector device; wherein the Risperidone microspheres are suspended in a non-aqueous liquid vehicle is medium chain triglycerides and easily injectable through 23G needle suitable for subcutaneous administration by patient himself or their caregiver.

In another embodiment, single component long-acting injectable non-aqueous formulation of poorly water soluble active pharmaceutical ingredient microspheres provides long term stability of a pharmaceutical composition in a non-aqueous liquid vehicle, thus eliminating any need for the patient or health care provider to add a pharmaceutically acceptable vehicle or carrier to the pharmaceutical composition prior to injection.

In another embodiment, single component long-acting injectable non-aqueous formulation of Risperidone microspheres provides long term stability of a pharmaceutical composition in a non-aqueous liquid vehicle, thus eliminating any need for the patient or health care provider to add a pharmaceutically acceptable vehicle or carrier to the pharmaceutical composition prior to injection.

In another embodiment, single component long-acting injectable non-aqueous formulation of Risperidone microspheres provides long term stability of a pharmaceutical composition in a non-aqueous liquid vehicle such as triglycerides; preferably medium chain triglycerides, thus eliminating any need for the patient or health care provider to add a pharmaceutically acceptable vehicle or carrier to the pharmaceutical composition prior to injection.

The present invention provides the risperidone microspheres composition in auto-injector have a volume of about volume of about 0.1 mL to about 10 mL, about 0.5 mL to about 7 mL, about 1 mL to about 7 mL, about 1 mL to about 5 mL; preferably the volume of about 5 mL or less, 4 mL or less, 3 mL or less, 2 mL or less, 1 mL or less, 0.5 mL or less; more preferably 0.1 mL, 0.15 mL, 0.2 mL, 0.25 mL, 0.3 mL. 0.35 mL, 0.4 mL, 0.45 mL, 0.5 mL, 0.55 mL, 0.6 mL, 0.65 mL, 0.7 mL, 0.75 mL, 0.8 mL, 0.85 mL, 0.9 mL, 0.95 mL or 1.0 mL.

The Injectable compositions comprising microparticle or microsphere preparations suspended in non aqueous vehicle are particularly susceptible to injectability problems. To be effective and pharmaceutically acceptable injectable microsphere suspension formulation preferably be sterile, stable, resuspendable, syringeable, injectable, isotonic and nonirritating. The number of characteristics includes syringeability, injectability, clogging, resuspendability and viscosity must be evaluated. Syringeability and injectability are key product performance parameters of any injectable dosage form. The syringeability includes such factors as ease of withdrawal of a product from a container, clogging of syringe needles and foaming tendencies, and accuracy of dose measurements. Injectability includes injection into the intended administration site, pressure or force required for injection, evenness of flow, and freedom from clogging (i.e., no blockage of the syringe needle). Syringeability and injectability can be affected by the microsphere particle size, density and viscosity of diluent. The particle size and viscosity of the microsphere injectable formulation should be optimized in order to facilitate mixing, resuspension of the particles with the vehicle, and to make the suspension easier to inject (i.e., low force on the syringe plunger). The viscosity can be measured by various methods known in the art but not limited to Ostwald type viscometer, suspended level viscometer, rotating viscometer, Anton-Paar's Modular Compact Rheometer 302 (MCR 302), Brookfield viscometer. The particle size and particle size distribution can be determined by various methods known in the art but not limited to Malvern's Mastersizer 3000, laser light diffraction (LD) method or using a Coulter counter.

The present invention provides the risperidone microspheres composition in auto-injector have a viscosity of about 100 cps or less, 90 cps or less, 80 cps or less, 70 cps or less, 60 cps or less, 50 cps or less, 40 cps or less, 30 cps or less, 20 cps or less, 10 cps or less.

The present invention provides the risperidone microspheres composition in auto-injector have a particle size distribution D10 in the range of 15 μm to 75 μm, D50 in the range of 40 μm to 120 μm and D90 in the range of 70 μm to 190 μm; preferably D10 in the range of 30 μm to 60 μm, D50 in the range of 60 μm to 100 μm and D90 in the range of 100 μm to 160 μm.

The present invention provides long-acting risperidone microsphere in medium chain triglycerides with improved syringeability and injectability via self-administration auto-injector device.

In one aspect of present invention, the self-administration auto-injector comprises risperidone micropsheres results in an effective amount of risperidone being released from the formulation to treat the risperidone sensitive diseases or disorders in mammals for an extended period of time.

In one aspect of present invention, the self-administration auto-injector comprises risperidone micropsheres results in an effective amount of risperidone being released from the formulation to treat the psychiatric diseases or disorders in mammals for an extended period of time.

To further illustrate the invention, the following examples are provided. It is to be understood that these examples are provided for illustrative purposes and are not to be construed as limiting the scope of the invention. It is to be further understood that, in the examples the functions of individual ingredients are sometimes listed for illustration purposes.

### EXAMPLES

### Example 1

Microspheres may be prepared by processes known in the art as described, e.g. U.S. Pat. No. 5,792,477.

Microspheres comprising a poly(lactide-co-glycolide) copolymer having dispersed therein 38.1% (w/w) Risperidone are obtained. The poly(lactide-co-glycolide) copolymer had a ratio of lactide:glycolide of 75:25.

### Example 2

The microspheres obtained from Example 1 were stored at 2-8° C. in non-aqueous carrier medium chain triglyceride (MCT), whereas the control sample was microsphere powder stored in glass vial, further stability of both test and control formulation will be investigated to determine the stability over an extended period of time.

The stability of the microspheres will be determined via, particle size, Risperidone Assay, Related substances, Burst Release and Molecular weight. Further the results for Initial analysis (To time point) is described in Table 1

TABLE 1
Stability study of Microsphere powder & microsphere
powder in MCT (MCT volume 0.8 ml)

| | | | | T0 Test | |
|---|---|---|---|---|---|
| Sr. | | T0 Control | | (Microsphere powder | |
| No. | Test Parameter | (Microsphere powder) | | in MCT) | |
| 1. | Product | White to off white | | White to off-white- | |
| | description | powder | | opaque suspension | |
| 2. | Assay | 96.6% | | 99.5% | |
| 3. | Related substances | | | | |
| | Impurity-A | ND | | ND | |
| | Impurity-B | ND | | ND | |
| | Impurity-C | 0.02 | | 0.00 | |
| | Impurity-E | ND | | ND | |
| | Max individual | 0.08 | | 0.04 | |
| | unknown | | | | |
| | impurity | | | | |
| | Total Impurity | 0.2 | | 0.13 | |
| 4. | Burst Release | 0.8% | | 0.7% | |
| 5. | Molecular weight | 108546 Daltons | | 97881 Daltons | |
| 6. | Particle size | | | | |
| | D10 | 46.45 | μm | 46.15 | μm |
| | D50 | 78.57 | μm | 79.43 | μm |
| | D90 | 125.77 | μm | 124.81 | μm |

ND: Not detected

### Example 3

Further second and third lot of microspheres manufactured as per process described in Example 1 were subjected to Stability study, Where the microspheres along with non-aqueous carrier medium chain triglyceride (MCT) were stored at 25° C./60% RH and at 2-8° C. and were analysed at Predetermined time points.
The stability of the microspheres is described in Table 2 and Table 3

TABLE 2
Stability study of microsphere powder in MCT
(MCT volume 0.8 ml)

| | | | | T3M | | T6M | | | | | |
|---|---|---|---|---|---|---|---|---|---|---|---|
| Sr. | Test | | | (25° C./ | | (25° C./ | | T3M | | T6M | |
| No. | Parameter | T0 Test | | 60% RH) | | 60% RH) | | (2-8° C.) | | (2-8° C.) | |
| 1. | Product | White to off-white-opaque suspension | | | | | | | | | |
| | description | | | | | | | | | | |
| 2. | Assay | 99.90% | | 96.2% | | 100.2% | | 96.4% | | 100.9% | |
| 3. | Related substances | | | | | | | | | | |
| | Impurity-A | 0.015% | | 0.02% | | ND | | 0.01% | | ND | |
| | Impurity-B | ND | | ND | | ND | | ND | | ND | |
| | Impurity-C | 0.02% | | BQL | | 0.02% | | 0.02% | | 0.02% | |
| | Impurity-E | ND | | ND | | BQL | | ND | | ND | |
| | Max | 0.03% | | 0.05% | | 0.08% | | 0.06% | | 0.08% | |
| | individual | | | | | | | | | | |
| | unknown | | | | | | | | | | |
| | impurity | | | | | | | | | | |
| | Total | 0.16% | | 0.20% | | 0.30% | | 0.19% | | 0.30% | |
| | Impurity | | | | | | | | | | |
| 4. | Burst | 2.6% | | 1.4% | | 1.4% | | 0.7% | | 0.9% | |
| | Release | | | | | | | | | | |
| 5. | Molecular | 97855 | | 93462 | | 85294 | | 112661 | | 104219 | |
| | weight | Daltons | | Daltons | | Daltons | | Daltons | | Daltons | |
| 6. | Particle size | | | | | | | | | | |
| | D10 | 56.02 | μm | 58.63 | μm | 54.80 | μm | 60.32 | μm | 55.00 | μm |
| | D50 | 94.76 | μm | 101.06 | μm | 100.50 | μm | 103.18 | μm | 99.40 | μm |
| | D90 | 144.67 | μm | 158.51 | μm | 163.10 | μm | 160.87 | μm | 158.60 | μm |

ND: Not detected
BQL: Below Quantification Limit

TABLE 3
Stability study of microsphere powder
in MCT (MCT volume 0.4 ml)

| Sr. | | | T3M |
|---|---|---|---|
| No. | Test Parameter | T0 Test | (25° C./60% RH) |
| 1. | Product | White to off-white- | |
| | description | opaque suspension | |
| 2. | Assay | 96.6% | 103.9% |

| | |
|---|---|
| 3. | Related substances |

| | | | |
|---|---|---|---|
| | Impurity-A | ND | ND |
| | Impurity-B | ND | ND |
| | Impurity-C | 0.13% | 0.02% |
| | Impurity-E | ND | ND |
| | Max individual | 0.04% | 0.08% |
| | unknown | | |
| | impurity | | |
| | Total Impurity | 0.11% | 0.29% |
| 4. | Burst Release | 0.7% | 0.6% |
| 5. | Molecular weight | 111265 Daltons | 96231 Daltons |

| | |
|---|---|
| 6. | Particle size |

| | | | | |
|---|---|---|---|---|
| D10 | 56.67 | μm | 56.17 | μm |
| D50 | 90.54 | μm | 90.28 | μm |
| D90 | 138.36 | μm | 138.35 | μm |

ND: Not detected

In view of the above stability details, it is evident that the proposed compositions are stable, and shall be evaluated further for suitable pre-clinical/clinical studies. The stability data results of both real time and accelerated conditions were found to be satisfactory.

We claim:

1. An self-administration injection device, comprising:
drug reservoir of liquid injectable composition comprising,
(i) risperidone microspheres and
(ii) vehicle;
wherein the said injection device is auto-injector for single dose;
wherein said injection is long-acting.

2. The self-administration injection device as claimed in claim 1, wherein the composition is present in a form suitable for subcutaneous administration.

3. The self-administration injection device as claimed in claim 1, wherein said device provided as kit comprising container comprising said composition and instructions for use.

4. The self-administration injection device as claimed in claim 1, wherein the vehicle is non-aqueous.

5. The self-administration injection device as claimed in claim 4, wherein the non-aqueous vehicle comprises of triglycerides.

6. The self-administration injection device as claimed in claim 1, wherein the risperidone microspheres suspended in triglycerides such as medium chain triglycerides.

7. The self-administration injection device as claimed in claim 1, wherein said composition has a viscosity is less than 100 cps.

8. The self-administration injection device as claimed in claim 1, wherein the volume of injection at least less than 5 ml, less than 3 ml, less than 2 ml or less than 1 ml.

9. The self-administration injection device as claimed in claim 1, wherein said device is digitally configured.

10. The self-administration injection device as claimed in claim 1, wherein the composition is used for treating risperidone sensitive diseases or disorders in mammals.
